(19) United States
(12) Patent Application Publication
(10) Pub. No.: US 2016/0178614 A1
(43) Pub. Date: Jun. 23, 2016

(54) CELL-BASED CONTROL AND METHOD

(71) Applicant: SIEMENS HEALTHCARE DIAGNOSTICS INC., Tarrytown, NY (US)

(72) Inventors: Michael Pugia, Granger, IN (US); Karen L. Marfurt, Edwardsburg, MI (US)

(73) Assignee: Siemens Healthcare Diagnostics Inc., Tarrytown, NY (US)

(21) Appl. No.: 14/909,449

(22) PCT Filed: Jul. 30, 2014
(86) PCT No.: PCT/US14/48884

§ 371 Date: Feb. 1, 2016

Related U.S. Application Data
(60) Provisional application No. 61/861,768, filed on Aug. 2, 2013.

(51) Int. Cl. G01N 33/50

(52) U.S. Cl. CPC G01N 33/5023; G01N 33/5044

(57) ABSTRACT

Control cells include a stable intact cell and, on the surface of the stable intact cell, a plurality of different antigens. Each of the different antigens corresponds to an antigen of interest. The control cells are employed in methods of evaluating the efficacy of a biological procedure performed on a sample. The method comprises conducting the biological procedure in the presence of the control cells. The control cells are examined to determine the efficacy of the biological procedure.

### CROSS-REFERENCE TO RELATED APPLICATIONS

This application claims priority to U.S. Provisional Application Ser. No. 61/861,768 filed Aug. 2, 2013, which is incorporated herein by reference in its entirety.

### BACKGROUND

The invention relates to control cells and their use as controls in biological procedures involving cells of interest.

Cellular analysis is important in medical applications such as, for example, diagnosis of many diseases. Methods involving cellular analyses include, but are not limited to, cell separation methods, cellular diagnostics that measure molecular and structural markers on the cell, cell functional assays that provide insight into the biochemical status of cells, cell activity assays that can measure cellular mechanism such as proliferation, cytotoxicity, viability, and apoptosis, for example. Cells can be detected by flow cytometers, microscopes, imaging methods, immunocytochemistry, in-situ hybridization, chromogen stains, receptor binding, proteomics methods, mass spectroscopy, RNA/DNA analysis, chemical analysis, immunoassay, automated assay, and other methods, for example. Control cells are employed in cellular analysis to assist in evaluating, for example, one or more of the functioning, accuracy, specificity, reliability, reproducibility, precision, morphology, efficiency of isolation or purification, activity, expression, proliferation, viability and other quality parameters of the cellular analysis including any equipment utilized in the analysis. The control cells may be used to measure the ability of the method to separate and/or isolate cells and/or to react cells with reagents such as those used in an assay. For example, control cells are employed as a daily control in rare cell detection assays.

In some known approaches involving control cells, fresh normal cells have been employed while in other known approaches abnormal cells have been used. In one specific example of a known approach, fluorescent labels are added to polymer beads, which can then be captured and used as controls. However, this type of control does not demonstrate that an assay, for example, an immunoassay, is functioning correctly, for example, similar to the target analyte(s) in an unknown sample. This is because the normal cell often does not have the antigens being detected in the diseased cells. Synthetic particles do not behave like nature cells and cannot test isolation efficiencies.

In another specific example of a known approach, whole cells have been used as the control material. For example, one approach uses SKBR cancer cells that naturally express the EpCAM antigen as a control for magnetic capture of cells by antibodies to EpCAM attached to magnetic particles. However, this type of control is limited to magnetic separation and does not demonstrate that the assay steps performed after the capture for other target proteins or antigens are functioning. For example, isolation of EpCAM expressing cells would not be detected when the rare cells are captured by filtration methods. Additionally, the control cells in this approach would not be performing as a functional control in the assay except to show that cells were isolated by filtration and that the detection mechanism for the labels, for example, imaging system for fluorescence was working.

In another specific example of a known control method, fluorescent labels can be attached to long chain lipophilic carbocyanine molecules that are available as soluble disulfonated and sulfopropyl derivative forms, which can be inserted into a cell. While this serves as a control for the ability of the microscope or flow cytometer to detect the fluorescence labels, it does not demonstrate that the assay is functioning as intended, for example, that antibodies have bound to the cell.

In another specific example of a known control method, serotype cells are preserved in which all the cells contain the antigen to be detected. These serotype cells can be used to functionally test that antibodies have bound to the cell. Unfortunately, one cell type rarely if ever expresses all the antigens needed to be detected at one time.

There is, therefore, a need for a cell-based control to test, among others, the operation of functional reactions, isolation techniques and detection methods in the analysis of cells of interest where multiple targets can be detected with a single control cell.

### SUMMARY

Some examples in accordance with the principles described herein are directed to a control cell comprising a stable intact cell and, on the surface of the stable intact cell, a plurality of different antigens. Each of the different antigens corresponds to an antigen of interest.

Some examples in accordance with the principles described herein are directed to a control cell comprising a cancer cell. A plurality of different antigens is on the surface of the cancer cell. Each of the different antigens corresponds to an antigen of interest. Each antigen of the plurality of antigens is attached to the surface of the cell by a protein linking group or a lipid linking group and the linking group is the same for the plurality of different antigens.

Some examples in accordance with the principles described herein are directed to methods of evaluating the efficacy of a biological procedure performed on a sample. The method comprises conducting the biological procedure in the presence of control cells. Each control cell comprises a stable intact cell having a surface on which a plurality of different antigens is attached. Each of the different antigens corresponds to an antigen suspected of being in a sample. The control cells are examined to determine the efficacy of the biological procedure.

### BRIEF DESCRIPTION OF THE DRAWINGS

The drawings provided herein are not to scale and are provided for the purpose of facilitating the understanding of certain examples in accordance with the principles described herein and are provided by way of illustration and not limitation on the scope of the appended claims.

FIG. 1 is a depiction of a stable intact cell having disposed on a surface thereof a plurality of antigens in an example in accordance with the principles described herein.

FIG. 2 is a depiction of a stable intact cell having disposed on a surface thereof a plurality of antigens in another example in accordance with the principles described herein.

### DETAILED DESCRIPTION OF SPECIFIC EMBODIMENTS

### Control Cells

Examples in accordance with the principles described herein are directed to a control cell having on its surface a plurality of antigens of interest for use in analyzing a sample of interest that is suspected of comprising a plurality of different antigens. Accordingly, examples in accordance with the principles described herein allow for multiplexed analysis of such samples. The antigens of interest are those antigens suspected of being in a sample that is subjected to analysis. A control cell in accordance with the principles described herein comprises a stable intact cell.

The term “stable” cell refers to a cell line that does not easily degrade to any significant degree and that is substantially similar from one cell to the next. The phrase “substantially similar” means that the characteristics of the cell from one cell to another cell do not change structurally by more than 10%. The term “intact” cell refers to a cell that is maintaining structural integrity such that the antigen on the cell is not compromised and an antibody continues to bind strongly and the cell shape is maintained.

The stable intact cells include normal cells, diseased cells (e.g., necrotic cells, and cells from diseased tissues), and mutated cells (e.g., cancer cells, and infected cells). Particular examples of stable intact cells, by way of illustration and not limitation, that may be employed in the control cells in accordance with the principles described herein include cells of epithelium origin (e.g., carcinomica, basil, transitional, squamous, cuboidal, and columnar), cells of endothelium origin, (e.g., placenta, large vessels and microvascular), cells of mesenchymal origin (e.g., stromal, stem cells, and sarcoma), immune cells/blood cells (e.g., monocytes, mononuclear cells, T cells, NK cells, B cells, stem cells, progenitor cells), fetal cells, myocytes, chondrocytes, fibroblasts, hepatocytes, keratinocytes, melanocytes, osteoblasts, preadipocytes, skeletal muscle cells, and smooth muscle cells, for example.

Cells of endothelium origin include, but are not limited to, endothelial cells such as, for example, Human Umbilical Vein Endothelial Cells (HUVEC), Human Large Vessel Endothelial Cells (HLVEC), Human Small Vessel Endothelial Cells (HSVEC); cells listed in the ATCC and other cell bank collection of primary and transformed endothelial cell lines of from a variety of species; for example.

Cells of epithelium origin include, but are not limited to, epithelial cells such as, for example, breast carcinoma cells (e.g., SKBR-3, SKBR-5, MDA, Hs 849.T, HTB30); non-small cell lung adenocarcinoma (N2228), brain astrocytoma (CCF-STTG1, SW 1783), cervix adenocarcinoma (HeLa), colon adenocarcinoma (H329, GH354, COLO 824) pancreatic adenocarcinoma (KLE, HPAC), renal cell adenocarcinoma (786-O, 769-P, MIA), prostate carcinoma cells (MDA PCa, LNCaP), pancreatic carcinoma cells (PaCa-2), bladder carcinoma (HT-1376, Hs 228.T), colorectal carcinoma, (SNU-C2B, LS411N), hepatocellular carcinoma, (SNU-449, C3A) pharynx squamous cell carcinoma (FaDu), transitional bladder carcinoma cells (SW 7801), melanoma (CHL-1), and others listed in the ATCC and other cell bank collections.

Cells of mesenchymal origin include, but are not limited to, fibrosarcoma connective tissue (15.T), lymphosarcoma lymph node (TE 175.T), adenocarcinoma, non-small cell lung cancer (H226), osteosarcoma (143.98.2) along with other stromal cells, stem cells, and sarcoma cells and other cells listed in the ATCC and other cell bank collections of cell lines of mesenchymal, stromal, stem cell, and sarcoma origin from a variety of species.

Cells of blood origin include, but are not limited to, leukemia bone marrow, myeloblast (KG-1), acute monocytic leukemia (THP-1), acute T cell leukemia (J.CaM1.6) acute promyelocytic leukemia (15 HL-60), lymphoma (1A2) and other cells listed in the ATCC and other cell bank collections of cell lines of blood origin from a variety of species.

Cells can originate from a variety of tissues such as, but not limited to, apidose tissue, bladder, blood/bone marrow/skeletal system, kidney, heart, umbilical cord, uterus, mammary tissue, lung, liver, brain, prostate, respiratory system, skin, digestive, endocrine, and lymphatic, for example.

A plurality of different antigens is present on the surface of the control cell. The number of different antigens on the surface of the control cell is determined by the nature of the sample, for example. The term “plurality” includes at least 2, or at least 3, or at least 4, or at least 5 or more different antigens. The number of antigens on the surface of the control cell may be in the range of 2 to about 100, or 2 to about 50, or 2 to about 25, or 2 to about 10, or 3 to about 50, or 3 to about 25, or 3 to about 10, or 4 to about 50, or 5 to about 50, for example. Each of the different antigens corresponds to an antigen suspected of being in a sample.

The samples may be biological samples or non-biological samples. Biological samples may be from a mammalian subject or a non-mammalian subject. Mammalian subjects may be, e.g., humans or other animal species. Biological samples include biological fluids such as whole blood, serum, plasma, sputum, lymphatic fluid, semen, vaginal mucus, feces, urine, spinal fluid, saliva, stool, cerebral spinal fluid, tears, and mucus, for example; biological tissue such as hair, skin, sections or excised tissues from organs or other body parts; for example. In many instances, the sample is whole blood, plasma, serum, urine or sputum. Non-biological samples include, but not limited to, environmental samples such as, e.g., waste streams, rivers, lakes, landfills, streams, marshes, dirt, samples from manufacturing processes, such as culture media and bioreactors, fermentation, and processed blood samples such as apheresis and cell enrichment processes, for example.

The term “antigens” refers to a moiety that binds specifically to a respective antibody and can be a variety of moieties of biological or medical interest. Examples include hormones, proteins, peptides, lectins, oligonucleotides, drugs, chemical substances, nucleic acid molecules, (e.g., RNA and/or DNA), glycoprotein, glyclolipids, enzymes, metabolites and particulate substances of biological origin, such as cells, viruses, and bacteria, for example. Protein antigens include, but are not limited to, immunoglobulins, cytokines, enzymes, hormones, cancer antigens, nutritional markers, tissue specific antigens, glycoprotein, and glyclolipids, for example.

Each antigen of the plurality of antigens is attached to the surface of the stable intact cell by means of a linking group. One or more linking groups may be employed. In some examples in accordance with the principles described herein the linking group coupling the antigens to the stable intact cell is the same for all of the antigens bound to the cell surface. In some examples, two or more, or three or more different linking groups may be employed. The number of different linking groups may be in the range of 2 up to the total number of antigens on the control cell. The nature of the linking group is dependent on one or more of the nature of the stable intact cell, and the nature of the antigen, for example.

The linking group is a protein capable of binding to a cell such that the bound protein can be conjugated to more than one antigen and the protein is capable of being fixed into a linked stable cell structure. Proteins can be conjugated by covalently attaching protein to another protein, ligand, lipid, glycan, or nucleic acid, for example. The protein or conjugate can bind to the cell through protein, ligand, receptor, membrane, nucleic acid interaction, or a conjugate interaction. The term “protein” includes synthetic peptide constructs such as polypeptides.

Many naturally-occurring proteins are capable of binding to a cell. For example, proteins with the cluster of differentiation (cluster of designation) are cell surface molecules capable of protein binding and useful for immunophenotyping of cells. By way of illustration and not limitation, proteins that bind cells include, but are not limited to, coagulation factors (e.g., factor X, factor VII, tissue factor), globular proteins (e.g., immunoglobulins), filamentous proteins (e.g., cytoplasmic filaments used in extracellular remodeling such as cytokeratin, vimentin collagen, elastin, and fibrin, for example), and receptor ligands (e.g., GPCR), for example.

A protein linking group may be attached to the surface of a stable intact cell in a number of different ways that depend on the nature of the protein, the nature of the stable intact cell, and the nature of the antigen, for example. In some examples in accordance with the principles described herein, a protein linking group is fixed to the surface of the stable intact cell. A fixing agent is employed to carry out this process of fixation. Fixing agents include, but are not limited to, substances that act to cross-link proteins and/or to disable proteolytic enzymes and prevent natural generation of fibrin. In some examples, the fixing agent is an aldehyde reagent (such as, e.g., formaldehyde, glutaraldehyde, and paraformaldehyde), a urea (such as, e.g., diazolidinyl urea or imidazolidinyl urea), an alcohol (such as, e.g., a C1 to C5 alkanol such as methanol or ethanol, for example), an oxidizing agent (such as, e.g., osmium tetroxide, potassium dichromate, chromic acid or potassium permanganate), for example.

Fixation of the cells immobilizes the cells and preserves cell structure and maintains the cells in a condition that closely resembles the cells in an in vivo-like condition and one in which the antigens of interest are able to be recognized by a specific binding partner for the antigen such as an antibody. The amount of fixative employed is that which preserves the cells but does not lead to erroneous results in a subsequent biological procedure such as an assay. The amount of fixative depends on one or more of the nature of the fixative and the nature of the cells, for example. In some examples, the amount of fixative is about 0.05% to about 0.15%, or about 0.05% to about 0.10%, or about 0.10% to about 0.15%, for example, by weight. Agents for carrying out fixation of the cells include, but are not limited to, cross-linking agents such as, for example, an aldehyde reagent (such as, e.g., formaldehyde, glutaraldehyde, and paraformaldehyde); an alcohol (such as, e.g., C1-C5 alcohols such as methanol, ethanol and isopropanol); a ketone (such as a C3-C5 ketone such as acetone); for example. The designations C1-C5 or C3-C5 refer to the number of carbon atoms in the alcohol or ketone. One or more washing steps may be carried out on the fixed cells using a buffered aqueous medium.

If necessary after fixation, the cell preparation is also subjected to permeabilization. In some instances, a fixation agent such as, for example, an alcohol (e.g., methanol or ethanol) or a ketone (e.g., acetone) also results in permeabilization and no additional permeabilization step is necessary. Permeabilization provides access through the cell membrane to antigens of interest. The amount of permeabilization agent employed is that which disrupts the cell membrane and permits access to the antigens. The amount of permeabilization agent depends on one or more of the nature of the permeabilization agent and the nature and amount of the cells, for example. In some examples, the amount of permeabilization agent is about 0.01% to about 10%, or about 0.1% to about 10%, for example. Agents for carrying out permeabilization of the cells include, but are not limited to, an alcohol (such as, e.g., C1-C5 alcohols such as methanol and ethanol); a ketone (such as a C3-C5 ketone such as acetone); a detergent (such as, e.g., saponin, Triton® X-100, and Tween®-20); for example. One or more washing steps may be carried out on the permeabilized cells using a buffered aqueous medium.

In an example in accordance with the principles described herein, the stable intact cell is of endothelium origin and the linking group comprises a coagulation protein. Particular examples of this approach include, by way of illustration and not limitation, a HUVEC cell with a Factor X linking group.

In another example in accordance with the principles described herein, the stable intact cell is of endothelium origin and the linking group comprises a filamentous protein. A particular example of this approach, by way of illustration and not limitation, includes an HUVEC cell with a cytokeratin linking group.

In another example in accordance with the principles described herein, the stable intact cell is of epithelium origin or mesenchymal and the linking group comprises an immunoglobulin. Particular examples of this approach include, but are not limited to, an immunoglobulin that binds to a filamentous protein, for example, a cytokeratin or a vimentin. Cytokeratins are proteins of keratin-containing intermediate filaments found in the intracytoplasmic cytoskeleton of epithelial tissue. Vimentin is often used as a marker of mesenchymally-derived cells or cells undergoing an epithelial-to-mesenchymal transition (EMT) during both normal development and metastatic progression.

In another example in accordance with the principles described herein, the stable intact cell is an immune cell and the linking group comprises an immunoglobulin. Particular examples of this approach include, but are not limited to, an immunoglobulin that binds to an FC receptor.

In some examples, antigens may be conjugated to the protein linking group by the reaction of a reactive functionality of the antigen with a reactive functionality of the protein linking group thereby forming a covalent bond linking the antigen to the protein. Reactive functionalities may occur naturally on the antigen and/or the protein or a reactive functionality may be introduced into the antigen and/or the protein by synthetic means. Such reactive functionalities include, but are not limited to, amine groups, hydroxyl groups, thiol groups, disulfide groups, and carboxyl groups, for example. In the linking process at least one of the reactive functionalities is activated with an activating agent. Some particular examples of reagents for coupling antigens to proteins, by way of illustration and not limitation, include glutaraldehyde (for coupling of amine groups to amine groups); carbodiimide (e.g., 1-ethyl-3-[3-dimethylaminopropyl]carbodiimide) (EDC or EDA) (for coupling carboxyl groups to amine groups); maleimide (e.g., sulfosuccinimidyl-4-(N-maleimidomethyl)cyclohexane-1-carboxylate (Sulfo-SMCC)), m-maleimidobenzoyl-N-hydroxysuccinimide ester (MBS) and sulfo-MBS (for coupling amine groups to sulfhydryl groups); dityrosine; N-(p-maleimidophenyl)isocyanate (PMPI) (for coupling sulfhydryl groups and hydroxyl groups), N-hydroxysuccinimide, N-succinimidyl 3-(2-pyridyldithio)propionate (for coupling of amine groups to thiol groups); for example.

In addition to covalent bonding of the protein and the antigen, non-covalent bonding that is substantially irreversible under the conditions of use of the control cells may he employed. For example, a small molecule may be attached to either the protein or the antigen and a binding partner for the small molecule may be attached to the other of the protein or the antigen. Small molecule-binding partner combinations are discussed in more detail below.

As mentioned above the linking group conjugate may comprise a lipid, which can insert into the hydrophobic membrane surfaces of cells. Lipids may be conjugated with antigens using techniques and reagents that are similar to those described above for the conjugation of antigens to proteins. In this manner proteins covalently bound to lipids may be formed.

In addition, non-covalent binding may be employed for binding of antigens to the lipid. For example, one method is KODE™ technology that may be used to covalently bind a small molecule such as, e.g., biotin, to the lipid. The KODE™ reagent consists of three parts: a functional component, e.g., a small molecule such as biotin, a spacer, and a diacyl lipid. In one example of a KODE™ reagent, a small molecule is conjugated to a maleimide-bearing carboxymethylglycine, which is conjugated to an activated adipate derivative of dioleoylphosphatidylethanolamine. KODE™ reagents disperse in water for biocompatibility and spontaneously and stably incorporate into cell membranes. Cells that are modified using a KODE™ reagent remain intact. In a particular example, biotin is employed as the small molecule and antigens are attached to streptavidin. The antigens of interest become bound to the functionalized lipid reagent, which then may be incorporated into astable intact cell to produce a control cell in accordance with the principles described herein having a plurality of different antigens on the surface of the control cell.

In some examples, the small molecule referred to herein has a molecular weight less than about 2000, or less than about 1500, or less than about 1000, or less than about 500, or less than about 400, or less than about 300, for example. Examples of small molecules, by way of illustration and not limitation, include biotin, digoxin, digoxigenin, 2,4-dinitrophenyl, fluorescein, rhodamine, small peptides (meeting the aforementioned molecular weight limits), vitamin B12 and folate, for example. Examples of small molecule-binding partner for the small molecule pairs, by way of illustration and not limitation, include biotin-binding partner for biotin (e.g., avidin, streptavidin and antibody for biotin), digoxin-binding partner for digoxin (e.g., antibody for digoxin), digoxigenin-binding partner for digoxigenin (e.g., antibody for digoxigenin), 2,4-dinitrophenyl and binding partner for 2,4-dinitrophenyl (e.g., antibody for 2,4-dinitrophenyl), fluorescein-binding partner for fluorescein (e.g., antibody for fluorescein), rhodamine-binding partner for rhodamine (e.g., antibody for rhodamine), peptide-binding partner for the peptide (e.g., antibody for the peptide), analyte-specific binding partners (e.g., intrinsic factor for B12, folate binding factor for folate), for example.

In some examples in accordance with the principles described herein, the linking group is a synthetic construct. Such synthetic constructs include, but are not limited to, synthetic proteins, synthetic lipids, synthetic peptides, glycan and nucleic acids, for example.

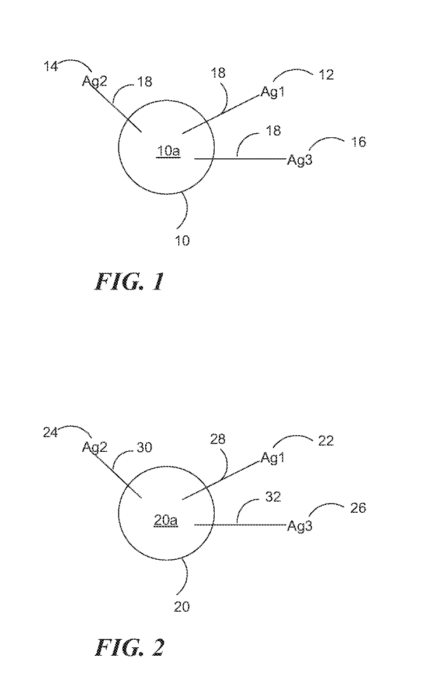

One example of a control cell in accordance with the principles described herein is depicted in FIG. 1. Referring to FIG. 1, cell 10 comprises antigen (Ag1) 12 that is bound to linking group 18, which in turn is attached to a surface 10a of cell 10. Surface 10a of cell 10 also comprises antigen (Ag2) 14 that is different from antigen 12 and that is attached to linking group 18, which in turn is attached to surface 10a. In this example, linking group 18 is the same moiety for each of the antigens attached. Surface 10a of cell 10 also comprises antigen (Ag3) 16 that is different from antigen 12 and antigen 14 and that is attached to linking group 18, which in turn is attached to surface 10a.

Another example of a control cell in accordance with the principles described herein is depicted in FIG. 2. Referring to FIG. 2, cell 20 comprises antigen (Ag1) 22 that is bound to linking group 28, which in turn is attached to a surface 20a of cell 20. Surface 20a of cell 20 also comprises antigen (Ag2) 24 that is different from antigen 22 and that is attached to linking group 30, which in turn is attached to surface 20a. Surface 20a of cell 20 also comprises antigen (Ag3) 26 that is different from antigen 22 and antigen 24 and that is attached to linking group 32, which in turn is attached to surface 20a. In this example, linking groups 28, 30 and 32 are different moieties for each of the antigens attached.

In a particular example in accordance with the principles described herein, a control cell comprises a cancer cell with a plurality of different antigens on the surface of the cancer cell. Each of the different antigens corresponds to an antigen suspected of being in a sample. Each antigen of the plurality of antigens is attached to the surface of the cell by a protein linking group or a lipid linking group and the linking group is the same for the plurality of different antigens.

Control cells in examples in accordance with the principles described herein may be stored in a suitable medium until use. The medium may be, but is not limited to, an aqueous medium, which may be solely water or may include from 0.1 to about 40 volume percent of a cosolvent such as an organic solvent (e.g., an alcohol or an ether). The pH for the medium will be in the range of about 4 to about 11, or in the range of about 5 to about 10, or in the range of about 6.5 to about 9.5, for example. Various buffers may be used to achieve the desired pH and maintain the pH during the assay. The medium may also comprise one or more of a preservative, a salt, a plasma protein, a protease inhibitor, a cell culture medium component, and a surfactant, for example. Any of the above materials, if employed, is present in a concentration or amount sufficient to achieve a desired effect or function. For example, a preservative is employed in an amount to achieve the desired preservative effect or function. The control cells may be stored at a temperature of about 2° C. to about 80° C., or about 2° C. to about 60° C., or about 2° C. to about 40° C., or about 2° C. to about 20° C., or about 5° C. to about 80° C., or about 5° C. to about 60° C., or about 5° C. to about 40° C., or about 5° C. to about 20° C., for example.

### Methods of Using Control Cells

Control cells in accordance with the principles described herein may be employed in any procedure or technique in which known control cells are used. Some examples in accordance with the principles described herein are directed to methods of evaluating the efficacy of a biological procedure performed on a sample. The methods comprise conducting the biological procedure in the presence of control cells. Each control cell comprises a stable intact cell having a surface on which a plurality of different antigens is attached. Each of the different antigens corresponds to an antigen of interest. The control cells are examined to determine the efficacy of the biological procedure. The phrase “efficacy of a biological procedure” refers to evaluating, for example, one or more of the functioning, accuracy, specificity, reliability, reproducibility, precision, morphology, efficiency of isolation or purification, activity, expression, proliferation, viability and other quality parameters of the cellular analysis including any equipment utilized in the analysis.

The biological procedures are those that involve cellular analysis; the biological procedures may include, but are not limited to, cell separation methods, assays, detection methods (for example, fluorescent imaging), and amplification methods, for example.

In one example in accordance with the principles described herein, control cells are used to measure the ability of a method to separate and/or isolate cells. Cell filtration for the separation of cells using a porous matrix is used to sort cells by size and, in most instances, such filtration methods allow for the extraction of cells following separation. Both microfluidic post and microfluidic membrane methods are used in these filtration approaches. Such filtration techniques include, but are not limited to, microfiltration, ultrafiltration, centrifugation, capillary flow or cross-flow filtration, for example. In a cell isolation technique, a porous or non-porous matrix is employed to assist in the separation and isolation of the various types of cells that may be present in a sample. Various cell types are differentiated from one another by size and/or the presence of different antigens on the cells in a sample.

Cells removed, released and/or collected from a surface of a porous matrix are subjected to further analysis. In some examples, analysis may be directed towards the four main biochemical classes, which are carbohydrates, lipids, proteins, and nucleic acids. Examples of analytic methods include, by way of illustration and not limitation, molecular methods such as next generation sequencing, polymerase chain reaction (PCR), microarray analysis, immunoassay techniques such as, for example, sandwich immunoassays and competitive immunoassays, and standard biochemical methods such as, for example, electrophoresis, chromatography, mass spectroscopy, spectroscopy, microfluidics, and microscopy. Control cells in accordance with the principles described herein may be employed in each of the above analytic methods to evaluate the efficacy of those methods.

In some examples, extracted or removed cells can be used to determine biomarkers on the cells. A biomarker is a moiety that facilitates the characterization or identification of a cell type and may also be referred to as an antigen. Proteins, RNA, DNA or cell components, for example, may be measured. Measurement of RNA in cell extracts may be carried out using a sensitive fluorescent nucleic acid stain for quantitating double-stranded DNA (dsDNA) and an ELISA plate reader. Other molecular methods that can be applied include, but are not limited to, RNA expression by microarrays, molecular probes such as b-DNA probes, sequencing, reverse transcription polymerase chain reaction (PCR), and quantitative real-time PCR.

Amplification methods for RNA may be employed for analysis of separated and isolated cell materials. Whole transcriptome amplification (WTA) uses reverse transcription polymerase chain reaction (qRT-PCR) with special enzymes and random-priming oligonucleotides to make cDNA and amplify a library of short, overlapping amplimers that are a very faithful representation of total cellular RNA.

Whole genome amplification methods for DNA may be employed in the analysis of separated and isolated cell materials. A Multiple Displacement Amplification (MDA) such as, for example, a REPLI-g® UltraFast kit (Qiagen, Inc., Valencia Calif.) or other commercially available kit may be employed. The MDA method uses DNA polymerase, buffers, and reagents for whole genome amplification. The average product length is typically greater than 10 kilobases (kb), with a range between 2 kb and 100 kb.

Select gene amplification methods for DNA by PCR may be employed in the analysis of separated and isolated cell materials. In this case a primer is used to cover a gene of interest or to cover panels of genes for a given disease state. In this approach, successful differentiation of wild type and mutant cell lines may be achieved at as little as ≧16 cells on a membrane by PCR amplification and sequencing using VERSANT® technology (Bayer HealthCare LLC, Berkeley, Calif.) or TRUGENE® technology (Bayer HealthiCare LLC).

The PCR approach to amplification is limited to specific regions of DNA, but allows a much lower (about 1% to about 0.01%) purity of rare cells to normal cells. This makes the PCR method attractive as secondary amplification method after whole-genome amplification (WGA) or whole transcriptome amplification (WTA). The first amplification (WGA or MTA) generates sufficient materials for detection and the second amplification (PCR) adds specificity to the use of lower DNA/RNA purity in the detection method. In one approach, a cell extract undergoes MDA pre-amplification followed by reverse transcriptase-PCR amplification, fragmentation, library preparation and size selection, cleanup, and finally sequencing.

In some examples, the cells are of different cell populations. In many instances the cells are rare cells, which are those cells that are present in a sample in relatively small quantities when compared to the amount of non-rare cells in a sample. In some examples, the rare cells are present in an amount of about 10⁻⁸% to about 10⁻²% by weight of a total cell population in a sample suspected of containing the rare cells. The rare cells may be, but are not limited to, malignant cells such as malignant neoplasms or cancer cells; circulating endothelial cells; circulating epithelial cells; fetal cells; immune cells (B cells, T cells, macrophages, NK cells, monocytes); stem cells; nucleated red blood cells (normoblasts or erythroblasts); and immature granulocytes; for example.

Non-rare cells are those cells that are present in relatively large amounts when compared to the amount of rare cells in a sample. In some examples, the non-rare cells are present in an amount of about 10²% to about 10⁸% by weight of a total cell population in a sample suspected of containing non-rare cells and rare cells. The non-rare cells may be, but are not limited to, white blood cells, platelets, and red blood cells, for example.

As mentioned above, control cells in accordance with the principles described herein may be employed to test the operation of functional reactions such as, for example, antigen-antibody reactions. Such reactions are typically involved in assays. The nature of the reagents employed is dependent on the particular type of assay to be performed. The assay may be an immunoassay or a non-immunoassay. Various assay methods are discussed below by way of illustration and not limitation.

In many embodiments the reagents comprise at least one antibody specific for an antigen on the cell that is characteristic of the cell, that is, the antigen is known to be associated with the particular cell in question. This assay is generally referred to as an immunoassay as distinguished from assays that do not utilize an antibody, which are referred to as non-immunoassays. By the phrase “antibody for an antigen” is meant an antibody that binds specifically to the antigen and does not bind to any significant degree to other substances that would distort the analysis for the particular antigen.

Antibodies specific for an antigen for use in immunoassays to identify cells can be monoclonal or polyclonal. Such antibodies can be prepared by techniques that are well known in the art such as immunization of a host and collection of sera (polyclonal) or by preparing continuous hybrid cell lines and collecting the secreted protein (monoclonal) or by cloning and expressing nucleotide sequences or mutagenized versions thereof coding at least for the amino acid sequences required for specific binding of natural antibodies.

Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, and Fab′, for example. In addition, aggregates, polymers, and conjugates of immunoglobulins or their fragments can be used where appropriate so long as binding affinity for a particular molecule is maintained.

Other reagents are included in the assay medium depending on the nature of the assay to be conducted. Such assays usually involve reactions between binding partners such as an antigen on a cell and a corresponding antibody or the binding between an antibody and a corresponding binding partner such as a second antibody that binds to the first antibody. The antibody and the antigen are members of a specific binding pair (“sbp member”), which is one of two different molecules, having an area on the surface or in a cavity, which specifically binds to and is thereby defined as complementary with a particular spatial and polar organization of the other molecule. The members of the specific binding pair will usually be members of an immunological pair such as antigen-antibody and hapten-antibody, although other specific binding pairs include, for example, biotin-avidin, hormones-hormone receptors, enzyme-substrate, nucleic acid duplexes, IgG-protein A, and polynucleotide pairs such as DNA-DNA, DNA-RNA.

Specific binding involves the specific recognition of one of two different molecules for the other compared to substantially less recognition of other molecules. The molecule that specifically binds to another molecule may be referred to as a specific binding partner for the other molecule. On the other hand, non-specific binding involves non-covalent binding between molecules that is relatively independent of specific surface structures. Non-specific binding may result from several factors including hydrophobic interactions between molecules. In many embodiments of assays, preferred binding partners are antibodies and the assays are referred to as immunoassays. Assays can be performed either without separation (homogeneous) or with separation (heterogeneous) of any of the assay components or products. Heterogeneous assays usually involve one or more separation steps and can be competitive or non-competitive.

Immunoassays may involve labeled or non-labeled reagents. Immunoassays involving non-labeled reagents usually comprise the formation of relatively large complexes involving one or more antibodies. Such assays include, for example, immunoprecipitin and agglutination methods and corresponding light scattering techniques such as, e.g., nephelometry and turbidimetry, for the detection of antibody complexes. Labeled immunoassays include, but are not limited to, chemiluminescence immunoassays, enzyme immunoassays, fluorescence polarization immunoassays, radioimmunoassay, inhibition assay, induced luminescence, and fluorescent oxygen channeling assay, for example.

As mentioned above, assays can be performed either without separation (homogeneous) or with separation (heterogeneous) of any of the assay components or products. Homogeneous immunoassays are exemplified by the EMIT® assay (Siemens Healthcare Diagnostics Inc., Deerfield, Ill.) disclosed in Rubenstein, et al., U.S. Pat. No. 3,817,837, column 3, line 6 to column 6, line 64; immunofluorescence methods such as those disclosed in Ullman, et al., U.S. Pat. No. 3,996,345, column 17, line 59, to column 23, line 25; enzyme channeling immunoassays (“ECIA”) such as those disclosed in Maggio, et al., U.S. Pat. No. 4,233,402, column 6, line 25 to column 9, line 63; the fluorescence polarization immunoassay (“FPIA”) as disclosed, for example, in, among others, U.S. Pat. No. 5,354,693; and enzyme immunoassays such as the enzyme-linked immunosorbent assay (“ELISA”). Exemplary of heterogeneous assays are the radioimmunoassay, disclosed in Yalow, et al., J. Clin. Invest. 39:1157 (1960). The relevant portions of the above disclosures are all incorporated herein by reference.

Other enzyme immunoassays are the enzyme modulate mediated immunoassay (“EMMIA”) discussed by Ngo and Lenhoff, FEBS Lett. (1980) 116:285-288; the substrate labeled fluorescence immunoassay (“SLFIA”) disclosed by Oellerich, J. Clin. Chem. Clin. Biochem. (1984) 22:895-904; the combined enzyme donor immunoassays (“CEDIA”) disclosed by Khanna, et al., Clin. Chem. Acta (1989) 185:231-240; homogeneous particle labeled immunoassays such as particle enhanced turbidimetric inhibition immunoassays (“PETINIA”), particle enhanced turbidimetric immunoassay (“PETIA”), etc.; and the like. Other assays include the sol particle immunoassay (“SPIA”), the disperse dye immunoassay (“DIA”); the metalloimmunoassay (“MIA”); the enzyme membrane immunoassays (“EMIA”); luminoimmunoassays (“LIA”); and so forth. Other types of assays include immunosensor assays involving the monitoring of the changes in the optical, acoustic and electrical properties of the present conjugate upon the binding of analyte. Such assays include, for example, optical immunosensor assays, acoustic immunosensor assays, semiconductor immunosensor assays, electrochemical transducer immunosensor assays, potentiometric immunosensor assays, amperometric electrode assays.

In many of the assays discussed herein, a label is employed; the label is usually part of a signal producing system (“sps”). The nature of the label is dependent on the particular assay format. An sps usually includes one or more components, at least one component being a detectable label, which generates a detectable signal that relates to the amount of bound and/or unbound label, i.e. the amount of label bound or not bound to the analyte being detected or to an agent that reflects the amount of the analyte to be detected. The label is any molecule that produces or can be induced to produce a signal, and may be, for example, an enzyme, a fluorescer, a chemiluminescer, a photosensitizer, or a radiolabel. Thus, the signal is detected and/or measured by detecting enzyme activity, luminescence, light absorbance or radioactivity, respectively.

Suitable labels include, by way of illustration and not limitation, enzymes such as alkaline phosphatase, glucose-6-phosphate dehydrogenase (“G6PDH”), β-galactosidase, and horseradish peroxidase; ribozyme; a substrate for a replicase such as QB replicase; promoters; dyes; fluorescers, such as fluorescein, isothiocyanate, rhodamine compounds, phycoerythrin, phycocyanin, allophycocyanin, o-phthalaldehyde, and fluorescamine; complexes such as those prepared from CdSe and ZnS present in semiconductor nanocrystals known as Quantum Dots; chemiluminescers such as luminal and isoluminol; sensitizers; coenzymes; enzyme substrates; radiolabels such as ¹²⁵I, ¹³¹I, ¹⁴C, ³H, ⁵⁷Co and ⁷⁵Se; particles such as latex particles, carbon particles, metal particles including magnetic particles, e.g., chromium dioxide (CrO₂) particles, and the like; metal sol; crystallite; liposomes; cells, etc., which may be further labeled with a dye, catalyst or other detectable group. The label can directly produce a signal and, therefore, additional components are not required to produce a signal. Numerous organic molecules, for example fluorescers, are able to absorb ultraviolet and visible light, where the light absorption transfers energy to these molecules and elevates them to an excited energy state. This absorbed energy is then dissipated by emission of light at a longer wavelength. Other labels that directly produce a signal include radioactive isotopes and dyes.

Alternately, the label may need other components to produce a signal, and the signal producing system would then include all the components required to produce a measurable signal. Such other components may include substrates, coenzymes, enhancers, additional enzymes, substances that react with enzymatic products, catalysts, activators, cofactors, inhibitors, scavengers, metal ions, and a specific binding substance required for binding of signal generating substances. Some known assays utilize a signal producing system (sps) that employs first and second sps members. The designation “first” and “second” is completely arbitrary and is not meant to suggest any order or ranking among the sps members or any order of addition of the sps members in the present methods. The sps members may be related in that activation of one member of the sps produces a product such as, e.g., light, which results in activation of another member of the sps.

In some examples by way of illustration and not limitation, the assay is an immunocytochemistry technique, a direct fluorescent antibody test or a direct immunofluorescence test.

In the immunocytochemistry technique, a labeled antibody specific for an antigen on a cell is employed for each suspected different cell population. The labels are fluorescent labels and a different fluorescent label is employed for each different cell population such that multiple fluorescent-labeled antibodies may be employed in any one assay conducted on a cellular sample.

A fluorescent DNA stain such as, for example, 4′,6-diamidino-2-phenylindole, propidium iodide, ethidium bromide, SYBR® Green I, VISTRA™ GREEN, SYTO® GREEN, SYBR® Gold, YO-PRO-1™, TOTO-3™, TO-PRO-3™, NUCLEAR-ID™ Red, or Hoechst dye, may be employed to enhance contrast during microscopic examination of the cellular material. After staining, one or more washing steps may be carried out on the cells using a buffered aqueous medium. The cells are then examined using a fluorescent microscope and each of the different fluorescent labels is used in the direct detection of a respective cell in the different cell populations.

Alternatively, in the above procedure unlabeled antibodies may be employed and the respective antibodies are detected indirectly employing a specific binding partner for each of the respective antibodies where the specific binding members are labeled with a fluorescent label or an enzyme label (such as, e.g., thiol-specific antioxidant (TSA enzyme)), for example. The respective labels of the specific binding partners are detected by appropriate means. The specific binding partners may be, for example, an antibody specific for each of the respective unlabeled antibodies used for binding to a respective antigen of a cell.

### Examination of Control Cells Used in a Bbiological Procedure

As mentioned above, the efficacy of a biological procedure performed on a sample can be evaluated using control cells in accordance with the principles described herein. After the biological procedure is carried out in the presence of control cells, the control cells are examined to determine the efficacy of the biological procedure. The control cells are subjected to the same examination to which the cells of interest are subjected. The type of examination is determined by the type of biological procedure that is conducted.

In many examples the examination involves detection of a signal from a medium comprising the cells of interest or a medium that results from subjecting the cells of interest to a particular biological procedure. The presence and/or amount of the signal is related to the presence and/or amount of a particular antigen in the sample. The particular mode of detection depends on the nature of the signal producing system employed. As discussed above, there are numerous methods by which a label of a signal producing system can produce a signal detectable by external means. Activation of a signal producing system depends on the nature of the signal producing system members

Luminescence or light produced from any label can be measured visually, photographically, actinometrically, spectrophotometrically, such as by using a photomultiplier or a photodiode, or by any other convenient means to determine the amount thereof, which is related to the amount of antigen in the medium. The examination for presence and/or amount of the signal also includes the detection of the signal, which is generally merely a step in which the signal is read. The signal is normally read using an instrument, the nature of which depends on the nature of the signal. The instrument may be, but is not limited to, a spectrophotometer, fluorometer, absorption spectrometer, luminometer, and chemiluminometer, for example.

The phrase “at least” as used herein means that the number of specified items may be equal to or greater than the number recited. The phrase “about” as used herein means that the number recited may differ by plus or minus 10%; for example, “about 5” means a range of 4.5 to 5.5.

The following examples further describe the specific embodiments of the invention by way of illustration and not limitation and are intended to describe and not to limit the scope of the invention. Parts and percentages disclosed herein are by volume unless otherwise indicated.

### EXAMPLES

All chemicals may be purchased from the Sigma-Aldrich Company (St. Louis MO) unless otherwise noted.

Abbreviations:

mL=milliliter

μL=microliter

mg=milligram

μm=micron(s)

min=minute(s)

h=hour(s)

rpm=revolutions per minute

FBS=fetal bovine serum

HBSS=Hanks Balanced Salt Solution

IMDM=Iscove's Modified Dulbeccos Medium

PBS=phosphate buffered saline as AMBION® PBS pH7.4 Thermo Scientific product number 158-0020

HBSS—Hank's Balanced Salt Solution

K₃EDTA=potassium salt of ethylenediaminetetraacetate

FITC=fluorescein isothiocyanate

TR=TEXAS RED™

Ex=Example

neg=negative

pos=positive

ATCC=American Type Culture Collection

Cell culturing: Cells were grown in culture using the recommended growth media. Human breast cancer cell line SK-BR-3 (ATCC HTB-30) was grown in IMDM Modified (HYCLONE®) (Pierce/Thermo Scientific, Rockford Ill.) plus 15% FBS (HYCLONE®). Human lung cancer cell line NCI H226 (ATCC CRL-5826) was grown in 10% FBS+RPMI-1640 (HYCLONE®). Cell line primary human umbilical vein endothelial cells (HUVEC) (ATCC PCS-100-010) were grown in ENDOGRO® Basal Media (Millipore Corporation, Billerica Mass.). All of the following operations were carried out under strict aseptic conditions. Cells in 6 mL complete medium were dispensed into 75 cm² culture flasks, each containing 3 mL of complete medium. The culture was incubated at 37° C. in a suitable incubator with 5% CO₂ in air atmosphere. Cells were sub-cultured two or three times weekly. When the culture was confluent, culture medium was removed, the cell layer was washed with 5 mL of 1× PBS and treated with trypsin, and cell cultures were split.

Cell fixation: Cells were fixed for refrigerated storage by removed media from a 75 cm² flask of cells at greater than or equal to 80% confluency. The cells were treated with 5 ml of 0.05% Trypsin solution (HYCLONE®) and added to the 75 cm² flask, the contents of which were incubated at 37° C. for 5 to 10 min until the cell layer was dispersed (cells were observed under an inverted microscope). Next, 5 mL of 10% FBS+RPMI-1640 (Life Technologies, Rockville Md.) were added and the contents of flask were transferred to the tube. After centrifuging at 3000 rpm for 5 min, the contents were decanted. A wash of 10 mL HBSS (Life Technologies) was added and the contents were centrifuged again at 3000 rpm for 5 min and decanted.

Cells were fixed by adding 1.0 mL of 2% formaldehyde HBSS to suspend the cell and incubating at 2° C. to 8° C. overnight for about 20 h. The cells were subjected to centrifugation at 2000 rpm for 5 min. Liquid was removed by decantation and the cells were washed twice with 1 mL PBS decanting wash liquid each time. To the cells were added 0.5 mL PBS and 2 μL of FBS and the resulting material was stored at 2° C. to 8° C. Cells were intact for at least 30 days at 2° C. to 8° C. and were freeze-thawed to −20° C. once. Cells were counted by mixing 30 μL of trypan blue solution (0.4%) and 10 μL of cells and reading on a hemacytometer. Typically, cells were at 10⁵/mL.

Linking group: Protein linking groups were fixed to the cells by means of formaldehyde. Cells (20 μL) were added to 0.1 to 10 mg/mL of protein linking group and the mixture was incubated for 30 minutes. All cells were from the ATCC. Linking groups were from Sigma-Aldrich with the exception of Human Factor X which was purchase from Enzyme Research Laboratory, South Bend Ind. Following centrifugation at 3000 rpm for 5 min, supernatant was decanted and the residue was resuspended with 1 mL HBSS and washing was repeated. Formaldehyde HBSS (2%) was prepared by adding 0.625 mL of 16% formaldehyde to 4.75 mL HBSS. The results are summarized in Table 1.

TABLE 1
Results of cell linking group examples

| Ex- | | | | | |
|---|---|---|---|---|---|
| am- | | | Tissue | Linkage | |
| ple | Cell | Cell type | origin | group | Binding site |
| 1 | SBBR | epithelial | breast | IgG | cytokeratin |
| 2 | HUVEC | endothelium | placenta | Factor X | Lipid |
| | | | | | membrane |
| 3 | HUVEC | endothelium | placenta | cytokeratin | Lipid |
| | | | | | membrane |
| 4 | H266 | epithelial-to- | lung | IgG | vimentin |
| | | mesenchymal | | | |
| 5 | Immune | monocyte | blood | IgG | FC receptor |
| | cell | | | | |
| 6 | SBKR | epithelial | breast | Streptavidin: | Lipid |
| | | | | Biotin-lipid | membrane |

Cell control preparation: Blood samples of about 8 mL were collected from normal donor following an IRB approved protocol using blood CaltagMedsytems Collection Tubes (Caltag-Medsystems, Buckingham UK) containing K₃EDTA and TRANSFIX® solution (Cytomark, Buckingham UK). Alternatively, blood samples of about 8 mL were collected from normal donor following an IRB approved protocol using blood Collection Tubes from Becton Dickinson and Company, Franklin Lakes N.J., containing K₃EDTA and 0.45 ml TRANSFIX® solution was added within 15 min of collection.

Fixed cells in PBS (2% FBS) at 10⁴ cells/mL (5 μL) were added to the centrifuge tube containing diluted blood and the sample was inverted 5 times to make a cell control of 50 cells/blood tube. Expected value was corrected by % change from expected in blood tube volume and % change in cell count by hemacytometer from expected. The sample is stored at room temperature.

Cell measurement: The blood samples were filtered through a membrane having an average pore size of 8 μm according to a method disclosed in U.S. Patent Application Publication No. 2012/0315664, the relevant portions of which are incorporated herein by reference. During filtration, the sample on the membrane was subjected to a negative mBar, that is, a decrease greater than about −30 mBar from atmospheric pressure. The vacuum applied varied from 1 to −30 mBar as the volume of the sample reduces from during filtration. High pressure drops were allowable dependent on reservoir and sample volume and filtration rate. Just prior to filtration, a sample (7-10 mL) was transferred to a 50 mL Falcon® tube, which was filled to 20 mL with cold PBS. The Falcon® tubes were manually overturned twice and subjected to centrifugation for 10 min, at 400×g at 20° C. The diluted sample was placed into the filtration station without mixing and the diluted sample was filtered through the membrane. Following the filtration, the membrane was washed with PBS, and the sample was fixed with formaldehyde, washed with PBS, subjected to permeabilization using of 0.2% Triton® X100 in PBS and washed again with PBS.

The size of the control cell is substantially the same as the size of a rare cell. The control cells do not pass through the pores of the filtration device and were separated at 95% recovery for Examples 1 to 5. The isolated controls cells were measured by fluorescence imaging after antibody reactions with linking groups. Antibodies were labeled with fluorescent dye for detection. In Examples 1-4, when the linking group was considered as an antigen (Factor X or Immuneglobulin (Ig)), the antigen was detected by antibody in all cases. In Examples 1, 2 and 3 an antigen was also attached to the linking group. Fluorescein (FITC) was the used as the antigen. An antibody to FITC was able to detect the antigen in all cases. In Example 5, streptavidin was conjugated with multiple antigens and antibodies to these antigens were able to detect the antigens in all cases.

In Example 6, SBKR cells and a biotin-lipid conjugate were employed, but streptavidin was replaced with and antibody for biotin as the linking protein where the antibody for biotin was labeled with FITC as the antigen. Cells without antigen tagged lipid or antibody labeled with antigen were negative (not fluorescent in green) as expected (See Examples 1 and 2 in Table 2). Cells with antigen tagged lipid or antibody labeled with dye were positive for green as expected (See Example 3). Addition of a second antibody to IgG linked to Texas red fluorescent dye demonstrated that a second antigen (in this case the antigen was IgG) can be simultaneously detected. This method allows for testing a plurality of antibody reactions with one control material. Each antibody was labeled with distinct fluorescent dyes allowing for distinguishing one antibody from another. These reactions can be for different antigens. The results are summarized in Table 2.

TABLE 2
Multiplex cell control

| | | | Anti | Anti- | | | |
|---|---|---|---|---|---|---|---|
| | | | FITC | mono- | | | |
| | Lipid | Anti- | mono- | clonal | | | |
| | FSL- | Biotin | clonal | mouse | Red | Green | |
| Ex | Biotin | FITC | mouse | TR | Result | Result | Result |
| 1 | yes | no | no | no | neg | neg | Negative |
| | | | | | | | control |
| 2 | No | yes | no | no | neg | neg | Negative |
| | | | | | | | control |
| 3 | yes | yes | no | no | neg | pos | Positive |
| | | | | | | | control |
| 4 | yes | no | yes | yes | neg | neg | Negative |
| | | | | | | | control |
| 5 | yes | yes | yes | no | neg | pos | Positive |
| | | | | | | | control |
| | | | | | | | level 1 |
| 6 | yes | yes | yes | yes | pos | pos | Positive |
| | | | | | | | control |
| | | | | | | | level 2 |

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A control cell comprising:
a stable intact cell and
on the surface of the stable intact cell a plurality of different antigens, each of the different antigens corresponding to an antigen of interest.

2. The control cell according to claim 1 wherein each antigen of the plurality of antigens is attached to the surface by a linking group.

3. The control cell according to claim 2 wherein the linking group comprises a protein.

4. The control cell according to claim 3 wherein the protein is fixed to the surface.

5. The control cell according to claim 3 wherein the protein is the same for the plurality of attached antigens.

6. The control cell according to claim 2 wherein the stable intact cell is of endothelium origin and the linking group comprises a coagulation protein.

7. The control cell according to claim 2 wherein the stable intact cell is of epithelial origin and the linking group comprises a filamentous protein.

8. The control cell according to claim 2 wherein the linking group is an immunoglobulin.

9. The control cell according to claim 2 wherein the linking group comprises a lipid.

10. The control cell according to claim 1 wherein the cell is a diseased cell, a cancer cell or a normal cell.

11. A control cell comprising:
a cancer cell and
on the surface of the cancer cell a plurality of different antigens, each of the different antigens corresponding to an antigen of interest, wherein each antigen of the plurality of antigens is attached to the surface by a protein linking group or a lipid linking group and wherein the linking group is the same for the plurality of different antigens.

12. The control cell of claim 11 wherein the linking group is a protein selected from the group consisting of coagulation proteins and immunoglobulins.

13. A method for evaluating the efficacy of a biological procedure performed on a sample, the method comprising:
(a) conducting the biological procedure in the presence of control cells wherein each control cell comprises a stable intact cell having a surface on which a plurality of different antigens is attached wherein each of the different antigens correspond to an antigen suspected of being in a sample, and
(b) examining the control cells to determine the efficacy of the biological procedure.

14. The method according to claim 13 wherein the biological procedure is a cell separation method, an assay, an imaging method, or an amplification method.

15. The method according to claim 13 wherein each antigen of the plurality of antigens is attached to the surface by a protein linking group or a lipid linking group.

16. The method according to claim 15 wherein the linking group is the same for the plurality of different antigens.

17. The method according to claim 15 wherein the linking group is a protein linking group selected from the group consisting of coagulation proteins and immunoglobulins.

18. The method according to claim 15 wherein the stable intact cell is of endothelium origin and the linking group comprises a coagulation protein.

19. The method cell according to claim 15 wherein the stable intact cell is of epithelial origin and the linking group comprises a filamentous protein.

20. The method according to claim 13 wherein the stable intact cell is a cancer cell.
